(19) United States
(12) Patent Application Publication
(10) Pub. No.: US 2016/0114050 A1
(43) Pub. Date: Apr. 28, 2016

(54) Heparosan/Therapeutic Prodrug Complexes and Methods of Making and Using Same

(71) Applicant: The Board of Regents of the University of Oklahoma, Norman, OK (US)

(72) Inventors: Paul L. DEANGELIS, Edmond, OK (US); Rachel S. LANE, Bartlesville, OK (US)

(21) Appl. No.: 14/921,732

(22) Filed: Oct. 23, 2015

Related U.S. Application Data
(60) Provisional application No. 62/067,761, filed on Oct. 23, 2014.

(51) Int. Cl. A61K 47/48; A61K 31/555; A61K 33/24

(52) U.S. Cl. CPC A61K 47/4823; A61K 33/24; A61K 31/555

(57) ABSTRACT

Compositions, methods, and systems are disclosed for the development and use of heparosan, a natural polymer related to heparin, as a new therapeutic modifying agent or complexation vehicle which can modulate drug cargo pharmacokinetics and behavior within a mammalian patient. In certain non-limiting embodiments, the use of heparosan is complexed with anti-cancer drugs and the like, thus forming a prodrug for the purposes of increasing efficacy and reducing side effects compared to the parental drug alone.

### CROSS REFERENCE TO RELATED APPLICATIONS/INCORPORATION BY REFERENCE STATEMENT

The present application claims benefit under 35 USC §119 (e) of U.S. Ser. No. 62/067,761, filed Oct. 23, 2014. The entire contents of the above-referenced patent application are hereby expressly incorporated herein by reference.

### BACKGROUND

1. Field of the Presently Disclosed and/or Claimed Inventive Concept(s)

Without limiting the scope of the presently disclosed and/or claimed inventive concept(s), the background of the related art is described in connection with the use of sugar polymers and, more particularly, heparosan [HEP] as a therapeutic modifying and/or complexing agent.

The presently disclosed and/or claimed inventive concept(s) relates generally to the field of therapeutics and, more particularly, to the development of enhanced therapeutics through the use of modifying and/or complexing agents and, in particular but without limitation, natural polysaccharides and oligosaccharides such as heparosan with utility for platinum compounds employed in the treatment of diseases, especially cancer. A wide range of existing and near-term therapeutics has great potential, but many possess drawbacks that slow or prevent implementation for aiding human health. Fortunately, the physical, chemical, and/or biological nature of a promising drug candidate may sometimes be assisted or improved by modifying the parental drug.

2. Description of the Related Art

As described in Li et al, (2002), chemotherapy for cancer, and in particular for recurrent and metastatic disease, has had limited therapeutic effects; this is mostly due to dose-limiting toxicity. Limited aqueous solubility, in vivo instability, and nonselectivity of promising anticancer drug candidates have long been stumbling blocks in cancer drug development. In the past, much effort has been made to develop novel anticancer drug formulations that would ensure the injectability, stability, and safety of these drug candidates. In the mid-1970s, Ringsdorf (1975) proposed a polymer-drug conjugate model that could enhance the delivery of an anticancer drug to a tumor. He envisioned that when an anticancer drug is conjugated to a polymeric carrier, its pharmacological properties could be manipulated by changing the physicochemical properties of the polymer. For example, an insoluble drug can be made water-soluble by introducing solubilizing moieties into the polymer. Likewise, active targeting is possible if a targeting moiety is introduced into the polymer. It was later recognized that polymer-drug conjugates tend to have enhanced uptake and persist longer in tumors and malignant growths than in normal tissues, even in the absence of a targeting moiety. Coined by Maeda and Matsurnara as ‘the enhanced permeability and retention effect,’ or the EPR effect, the phenomenon is attributed to the greater permeability of disordered capillary endothelia in malignant tumors towards macromolecules than normal tissue, as well as the lack of functional lymphatics in solid tumors (Maeda and Matsumara (1989); Gerlowsk) and Jain (1986); Jain (1998)). This view is clearly supported by the electron microscopic observation that peripheral tumor vascular endothelium has quantitatively more fenestrations and open junctions than normal vessels (Roberts and Palade (1997)). In addition to the EPR effect, tumor cells show a higher degree of uptake of macromolecules by endocytosis than do normal cells, resulting from the enhanced metabolic activity of cancer cells.

Cisplatin (cisplatinum, platarnin, neoplatin, cismaplat or cis-diamminedichloroplatinum(II)), approved for use in testicular and ovarian cancer by the FDA since 1978 and bladder cancer since 1993, is being used as a chemotherapy agent for a broad range of cancers. However, cisplatin has been linked to serious side effects that include (but are not limited to) nephrotoxicity, peripheral neuropathy, and ototoxicity. In an effort to alleviate such detrimental secondary effec(s, cornplexation and/or conjugation of cisplatin to drug delivery systems (such as a carrier polymer) have been investigated. Several delivery strategies have been investigated that employ such a polymer, including micelles, liposomes, and nanoparticles. In addition to preventing a low molecular weight drug from being excreted quickly if it was utilized alone, the use of larger molecular weight polymer complexes with the drug can also help drive the trapping of the therapeutic by tumors. Also, in addition to cisplatin, other platinum (e.g., picoplatin; azane-2-methylpyridine-platinum(2+)-dichloride) or metal compounds and the like can be complexed.

Ion complexation of metal compounds (e.g., cisplatin, picoplatin) to the carrier or to the polymer backbone has been employed in the past. Typically, a stable complex is formed and isolated in vitro by the hand of man, which, after administration to the mammalian patient, then breaks down or releases the original (or similar) drug or drug candidate in the body. When molecules employ this paradigm, they typically are known as prodrugs. Polymers of different natures, such as polysaccharides and polypeptides, have been evaluated in this transient carrier or prodrug strategy. Some of the prior art that utilize various polymers in this manner include Maeda et al. (1993); Peng et al. (2011); Cai et al. (2008); Jeong et al. (2008); Zhang et al. (2000); Zhang et al. (2002); U.S. Pat. No. 8,088,412; U.S. Pat. No. 8,895,076; Schechter et al (1986); and US Patent Application Publication No. US 2013/0259944. However, none of the prior art references discloses the use of heparosan as a polymer in this manner.

Therefore, there is a need in the art for new and improved carrier technologies that overcome the disadvantages and the defects in the prior art. In the presently disclosed and/or claimed inventive concept(s), another polymer, heparosan, a distinct member of the glycosaminoglycan (GAG) polysaccharide family that includes HA, chondroitin, and heparin, is used as the carrier. Heparosan has multiple advantages over the other polymers, including (but not limited to): (a) less substantial biological interactions and degradation in the extracellular spaces, (b) a longer half-life in the bloodstream of healthy animals, (c) a natural biodegradation pathway to prevent accumulation in the tissues after the drug is deployed, (d) more flexibility in achievable polymer sizes, and/or (e) no observed immunogenicity.

### BRIEF DESCRIPTION OF THE SEVERAL VIEWS OF THE DRAWINGS

FIG. 1 is a schematic representation of the heparosan polymer and two non-limiting examples of production routes therefor.

FIG. 2 is a graphical representation of the pharmacokinetics (PK) of a radioactively-labeled heparosan-cargo complex in plasma in a Sprague Dawley rat model. Rats were injected intravenously (Panel A) or intramuscularly (Panel B) with ¹²⁵I-heparosan polymer (100 kDa mass) Bolton-Hunter at ‘Time 0,’ and at various times, blood was drawn, and the radioactivity in the plasma was measured. The data indicate that 100 kDa heparosan, the carrier molecule of the heparosan/drug complexes discussed herein, has a long lifetime (here, a half-life of approximately two to three days, depending on injection route in the mammalian bloodstream.

FIG. 3 is a graphical representation of the fate of a radioactive Bolton-Hunter heparosan-cargo complex in Sprague Dawley rats. Rats were injected intravenously with 100 kDa ¹²⁵I-heparosan polymer at ‘Time 0,’ and at various times, the radioactivity in blood (‘Plasma’), red and white blood cells (‘R&W BC’), organs (liver, kidney, spleen, heart, bladder, brain), and excreted waste (urine, feces) was measured. The data indicate that heparosan, the carrier molecule of the heparosan/drug complexes discussed herein, circulated in the plasma of the mammalian blood stream (shown with a white bar), did not accumulate in major organs (note: the low signal present is due to blood trapped in organs based on saline-perfused controls), and was excreted via normal pathways (i.e., urine, feces; shown with a black bar)

FIG. 4 is a pictorial representation showing heparosan is very stable in the mammalian bloodstream. The 100 kDa ¹²⁵I-heparosan Bolton-Hunter cargo complex was injected intramuscularly (M) or post intraperitoneally (P) into Sprague Dawley rats (Panel A) or subcutaneously (SC) into Cynologus monkeys (Panel B), and at various times, blood was withdrawn, and the plasma was isolated. The samples were deproteinized and analyzed by agarose gel (1.5%) electrophoresis and autoradiography. The presence or absence of degradation products was assessed over five or seven day periods in rats or monkeys, respectively. The arrow indicates the migration of the starting probe (lane S). The molecular weights of the starting probe and the polymer in plasma samples were equivalent even after multiple days, and the absence of smaller molecular weight products indicates the lack of heparosan degradation in the extracellular compartments.

FIG. 5 depicts the pharmacokinetics (PK) of a subcutaneous (SC) injection of 100 kDa heparosan Bolton-Hunter ¹²⁵I conjugate in plasma in a non-human primate model, the Cynologus monkey (performed by Xenometrics LLC, Stilwell, Kans.). The radioactivity in plasma was measured over time. Here, this heparosan-conjugate demonstrates an approximately eight day half-life.

FIG. 6 depicts analysis of pooled urine samples from 1 to 6 Days (D) post-subcutaneous (post-SC) injection of 100 kDa HEPtune-Bolton-Hunter (BH) ¹²⁵I complex in primate model by Normal Phase Thin Layer Chromatography and autoradiography. Defined synthetic standards of Bolton-Hunter (BH) with short chains of 1, 2, or 3 sugars (Hep₁, Hep₂, or Hep₃, respectively) as well as free Bolton-Hunter are run in parallel. The major steady state metabolite (marked with a *) appears to be GlcUA-BH a small fragment of the original large molecular weight complex.

FIG. 7 depicts the serological test results for potential induction of anti-heparosan antibodies against a heparosan-containing molecule (HEP-G-CSF, heparosan sugar polymer attached to the Granulocyte Colony Stimulating Factor (G-CSF) protein) in a rat model. A control pre-immune sera was collected, and then three animals (Rat #1, #2, and #3) were immunized and periodically boosted with HEP-G-CSF eight times over an approximately seven month period followed by periodic test bleeds. The various serum samples were tested in 96-well plates via ELISA. First, the wells of the plate were coated either with: (i) bovine serum albumin (BSA) to assess the background (‘Control’ shown in grey bars); or (ii) heparosan-BSA (HEP-BSA) conjugate (‘HEP’; shown in black bars), to measure any potential immune response. After blocking all the wells with BSA and washing thoroughly, 1 μl of the pre-immunization serum (the negative control Prebleed, ‘PreB’) or post-immunization serum (collected either on week 9 (Bleed #1; B#1), week 15 (B#2), or week 30 (B#3)) was incubated with wells (the rats had 3, 5, or 9 injections of test antigen, respectively, before these three bleeds). After washing the wells, the presence of any bound rat antibodies was determined by interaction with either an anti-rat IgG (Panel A) or an anti-rat lgM (Panel B) goat secondary antibody-horseradish peroxidase conjugate. After washing, a colorimetric substrate was then used to detect any signal at 405 nm. Each bar corresponds to an averaged triplicate well determination. Even after multiple boosts with the HEP-drug conjugate, no specific antibodies against heparosan were observed (i.e., no significant differences were observed between the signals of the HEP wells and the BSA control wells).

FIG. 8 is graphical representation showing synthesis of monodisperse heparosan polymers. Various batches of heparosan polymer were analyzed on a 1.2% agarose gel with Stains-All detection. The polymer size is readily controlled (as indicated by the different size bands of 27 kDa to 1,300 kDa from bottom to top). The tight bands indicate that the products have a narrow size distribution (polydispersity values (M_{w}/Mₙ) of 1.02 to 1.18; for reference, the value of an ideal monodisperse polymer is 1). L=HA standard ladder (Hyalose, LLC, Oklahoma City, Okla.). The size of the polymer affects its half-life in the bloodstream and its targeting to tumors. In addition, the Food & Drug Administration (FDA) regulatory hurdles for production and approval of therapeutics are lower for a more defined, monodisperse molecule in comparison to a less defined, polydisperse molecule.

FIG. 9 graphically depicts structures of cisplatin (drug is circled) complexed with heparosan tetrasaccharide (Panels A and B), a model of part of the longer heparosan chains. ln the presence of free chloride (Cl⁻), as found in bodily fluids, the drug is slowly released from the polymer (Panel C).

FIG. 10 is graphical representation demonstrating the stability of heparosan prodrug complexes (here with cisplatin) in water and the release of the drug in saline (Panel A) or physiological fluids (Panel B). A 300 kDa heparosan polymer/cisplatin complex was challenged for various times (4, 18, or 24 hours) at 37° C. with either (i) water, (ii) 0.1 M sodium chloride buffered with 10 mM HEPES, pH 7.2, or (iii) ultra-filtered human plasma. Size exclusion chromatography (Sephadex G-25; PD10 column, GE Healthcare Bio-Sciences, Pittsburgh, Pa.) was then employed to measure bound drug attached to long heparosan chains (polymer elutes in the column void volume) over time using the OPD assay with output of optical density at 706 nm.

FIG. 11 is a graphical representation of a plot depicting the percentage of cisplatin released from heparosan over time in each challenge condition, either a saline solution (NaCl) or human plasma, described in FIG. 10. Release was normalized to the pro-drug incubated in water at the same temperature and duration as the challenge condition. This data indicates an approximately 10-hour half-life for the prodrug in physiological fluids.

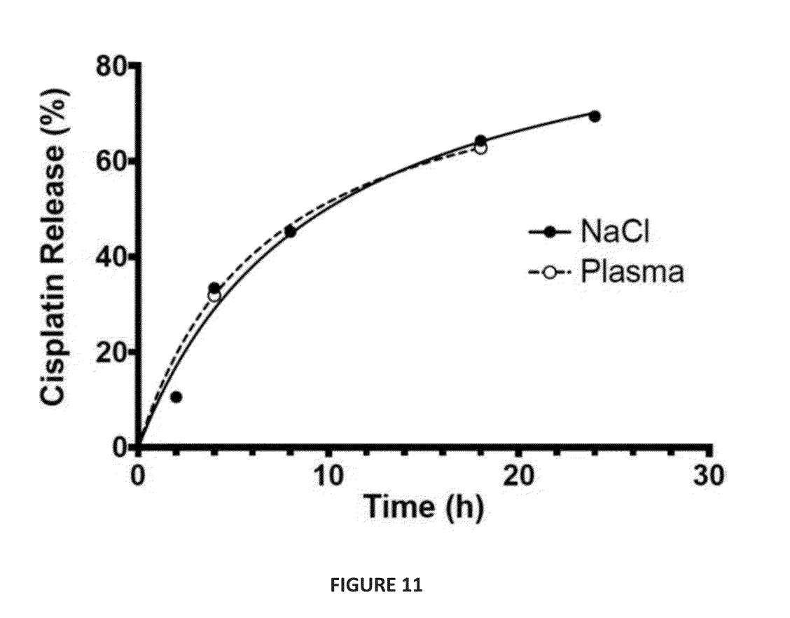

### DETAILED DESCRIPTION

Before explaining at least one embodiment of the presently disclosed inventive concept(s) in detail, it is to be understood that the presently disclosed inventive concept(s) is not limited in its application to the details of construction and the arrangement of the components or steps or methodologies set forth in the following description or illustrated in the drawings. The presently disclosed inventive concept(s) is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the presently disclosed inventive concept(s) shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, complexes and/or methods disclosed or otherwise contemplated herein can be made and executed without undue experimentation in light of the present disclosure. While the compositions, complexes, and methods of the presently disclosed and/or claimed inventive concept(s) have been described in terms of particular embodiments, it will be apparent to those of ordinary skill in the art that variations may be applied to the compositions, complexes, and/or methods as well as in the steps and/or in the sequence of steps of the methods described herein without departing from the concept, spirit, and scope of the presently disclosed and/or claimed inventive concept(s). All such similar substitutes and modifications apparent to those of ordinary skill in the art are deemed to be within the spirit, scope, and concept of the presently disclosed and/or claimed inventive concept(s).

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one,” The use of the term “or” in the claims is used to mean “and/or” unless explicitly indicated to refer to alternatives only or the alternatives are mutually exclusive, although the disclosure supports a definition that refers to only alternatives and “and/or:” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are riot to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example,

As used in this specification and claim(s), the words “comprising” (and any form of comprising, such as “comprise” and “comprises”), ‘having’ (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps,

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, and/or CAB. Continuing with this example, expressly included are combinations that contain repeats of one or more item or term, such as BB, AAA, AAB BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule can be biologically active through its own functionalities, or may be biologically active based on its ability to activate or inhibit molecules having their own biological activity.

As used herein, “substantially pure” means an object species is the predominant species present (i.e., on a molar basis it is more abundant than any other individual species in the composition); for example, in certain non-limiting embodiments, a substantially purified fraction is a composition wherein the object species comprises at least about 50 percent (on a molar basis) of all macromolecular species present. Generally, a substantially pure composition will comprise more than about 80 percent of all macromolecular species present in the composition, such as (but not limited to) more than about 85%, 90%, 95%, and 99%. In a particular, non-limiting embodiment, the object species may be purified to essential homogeneity (contaminant species cannot be detected in the composition by conventional detection methods) wherein the composition consists essentially of a single macromolecular species.

The term “patient” as used herein includes human and veterinary subjects. “Mammal” for purposes of treatment refers to any animal classified as a mammal, including human, domestic and farm animals, nonhuman primates, and any other animal that has mammary tissue.

“Treatment” refers to both therapeutic treatment and prophylactic or preventative measures. Those in need of treatment include, but are not limited to, individuals already having a particular condition or disorder as well as individuals who are at risk of acquiring a particular condition or disorder (e.g., those needing prophylactic/preventative measures). The term “treating” refers to administering an agent to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, or management of a disease and/or condition. The specific amount that is therapeutically effective can be readily determined by the ordinary medical practitioner, and can vary depending on factors known in the art, such as the type of disease/cancer, the patient's history and age, the stage of disease/cancer, and the co-administration of other agents.

A “disorder” is any condition that would benefit from treatment with the compositions disclosed herein. This includes chronic and acute disorders or diseases including those pathological conditions which predispose the mammal to the disorder in question.

The term “effective amount” refers to an amount of a biologically active molecule or complex or derivative thereof sufficient to exhibit a detectable therapeutic effect without undue adverse side effects (such as toxicity, irritation and allergic response) commensurate with a reasonable benefit/risk ratio when used in the manner of the inventive concept(s). The therapeutic effect may include, for example but not by way of limitation, inhibiting the growth of microbes and/or opportunistic infections. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the disease in conjunction with the pharmaceutical compositions of the presently disclosed and claimed inventive concept(s). This concurrent therapy can be sequential therapy, where the patient is treated first with one drug and then the other, or the two drugs are given simultaneously.

The terms “administration” and “administering,” as used herein will be understood to include all routes of administration known in the art, including but not limited to, oral, topical, transdermal, parenteral, subcutaneous, intranasal, mucosal, intramuscular, intraperitoneal, intravitreal and intravenous routes, including both local and systemic applications. In addition, the compositions of the presently disclosed and claimed inventive concept(s) (and/or the methods of administration of same) may be designed to provide delayed, controlled or sustained release using formulation techniques which are well known in the art.

The presently disclosed and/or claimed inventive concept(s) provides for the improvement and enhancement of therapeutics through the complexation thereof with a novel therapeutic modifying agent: heparosan, a natural polysaccharide related to heparin. Heparosan can be synthesized in a reproducible and defined manner. Heparosan is soluble in water, biocompatible, and relatively inert in the extracellular spaces within the mammalian body. It has multiple advantages over the other GAGs or polyglutamic acid polymers or plastic-like polymers; these advantages include (but are not limited to) at least one of the following: (a) less substantial biological interactions and degradation in the extracellular spaces, (b) longer half-life in the bloodstream of healthy animals, (c) more flexibility in achievable polymer sizes, and/or (d) no observed immunogenicity.

Certain carbohydrates play roles in forming and maintaining the structures of multicellular organisms in addition to more familiar roles as nutrients for energy. Glycosaminoglycans (GAGs) are long linear polysaccharides comprising disaccharide repeats that contain an amino sugar. GAGs are well known to be essential in vertebrates.

The GAG structures possess a significant number of negative groups and hydroxyl groups and are, therefore, highly hydrophilic. Depending on the tissue and cell type, some GAGs can be structural, adhesion, and/or signaling elements in humans. A few microbes also produce extracellular polysaccharide coatings called capsules that are composed of GAG chains and that serve as virulence factors (DeAngelis, 2002). The capsule assists in the microbe's evasion of host defenses such as phagocytosis and complement. As the microbial polysaccharide is identical or very similar to the host GAG, the antibody response to the microbe is either very limited or non-existent.

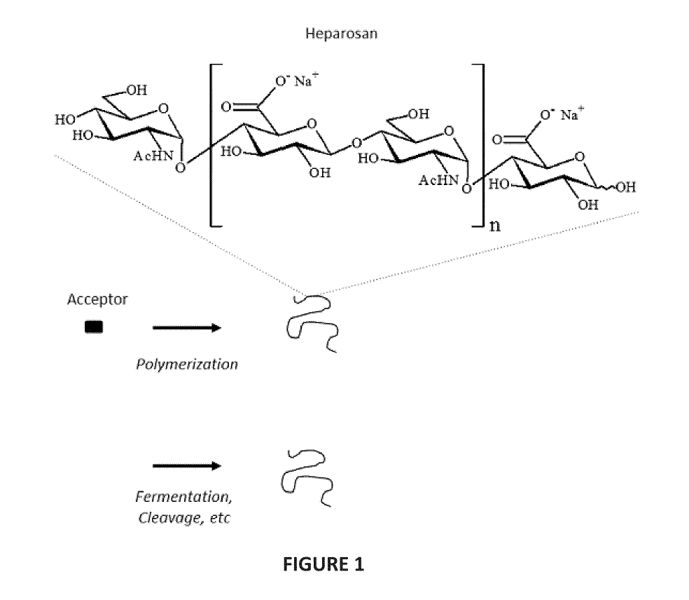

Heparosan (HEP) is also referred to in the art as N-acetylheparosan or unsulfated, unepimerized heparin, and possesses the structure [-4-GlcUA-beta-1,4-GlcNAc-alpha-1-]ₙ (FIG. 1). In humans, polymers of heparosan exist transiently, serving as a precursor to the more highly modified final products of heparan sulfate and heparin. The bacterial-derived enzymes used to produce heparosan for use in one embodiment of the presently disclosed and/or claimed inventive concept(s) synthesize heparosan as their final product. A single polypeptide, the heparosan synthase PrnHS1 of Pasteurella multocido Type D, polymerizes the heparosan sugar chain by transferring both GlcUA and GlcNAc. PmHS1 is a robust enzyme that efficiently makes polymers up to ˜1 MDa (1,000 kDa or ˜5,000 monosaccharide units) in vitro. Some P. multocida strains also have a second similar enzyme, PmHS2, that can polymerize heparosan. Likewise, some microbes related to Posteurella, called Avibacteria, have enzymes with similar sequences. PrnHS1 and PmHS2 are very active and stable in recombinant forms (including fusion proteins, truncations, mutants, or analogs and combinations thereof). In addition, chimeric recombinant enzyme versions combining the activities of PmHS1 and PrnHS2 can be used with varying levels of efficiency.

In Escherichia coli K5, at least two enzymes, KfiA, the alpha-GlcNAc transferase, and KfiC, the beta-GlcUA-transferase, (and perhaps KfiB, a protein of unknown function) work in concert to form the disaccharide repeat of heparosan. The E. coli enzyme complex is not as efficient as the PmHS1 enzyme in vitro, as it is more difficult to produce the long polymer chains with the E. coli enzyme complex. However, for the purpose of the presently disclosed and/or claimed inventive concept(s), it is intended and would be understood by one of ordinary skill in the art that any method which produces heparosan may be used (FIG. 1). It is not the method of producing heparosan that is determinative—rather, the presently disclosed and/or claimed inventive concept(s) is directed to the complexation of heparosan from any source or method of production (including, but not limited to, fermented heparosan produced by native or recombinant microbes, as well as chemoenzymatic syntheses or organic chemical syntheses) with a target molecule (i.e., the cargo, such as a drug or drug candidate), thereby providing for increased solubility in water, bioavailability and dwell time within the patient, based upon the utilization of heparosan as the carrier.

A key advantage to using heparosan in this manner is that it exhibits increased biostability and long half-life in the extracellular matrix when compared to other GAGs, such as hyaluronic acid and chondroitin. As with most compounds synthesized in the body, new molecules are typically made, and after serving their purpose, are broken down into smaller constituents for recycling. Table 1 describes some of the differences amongst the GAG family and other structurally related polymers.

TABLE 1
Various Activities/Features of Glycosaminoglycans & Related Polymers

| | Activities/Features | | | | | | | |
|---|---|---|---|---|---|---|---|---|
| | | | | Protein- | | | | |
| GAG | HAase¹? | HEPase²? | Lysosome³? | Active⁴? | Soluble⁵? | Mammalian⁶? | Half-life⁷? | |
| Heparosan | No | No | Yes | Not | Yes | Yes | ^{~}16-192 | hrs |
| | | | | reported | | | | |
| Hyaluronan (HA) | Yes | No | Yes | Yes | Yes | Yes | ^{~}2-5 | min |
| Chondroitin | Yes | No | Yes | Yes | Yes | Yes | ? | |
| Chondroitin sulfate | Yes | No | Yes | Yes | Yes | Yes | minutes* | |
| Dermatan sulfate | Yes | No | Yes | Yes | Yes | Yes | minutes* | |
| Heparan sulfate | No | Yes | Yes | Yes | Yes | Yes | 1-6 | hrs |
| Heparin | No | Yes | Yes | Yes | Yes | Yes | 1-6 | hrs |

| | | | | | | | |
|---|---|---|---|---|---|---|---|
| Keratan sulfate⁸ | No | No | Yes | Yes | Yes | Yes | ? |
| Chitin | No | No | Yes* | No* | No | No | N/A |
| Chitosan | No | No | Yes | Yes | Yes | No | ? |

¹substantially sensitive to cleavage by mammalian hyaluronidases (HAase);
²substantially sensitive to cleavage by mammalian heparanase (HEPase);
³substantially sensitive to cleavage by mammalian lysosomal glycosidases;
⁴capable of specifically and significantly interacting or binding with tested proteins or cells, resulting in biological outputs including adhesion, signaling, morphogenesis, proliferation, etc.;
⁵polysaccharide form soluble in water;
⁶a naturally occurring molecule in the mammalian tissues;
⁷approximate half-life of polysaccharide in the plasma after injection, based on inventor's experimental data for heparosan and the remainder from peer-reviewed literature;
⁸digested by keratanase;
*denotes a hypothetical answer not tested by direct experiment;
N/A, not applicable.

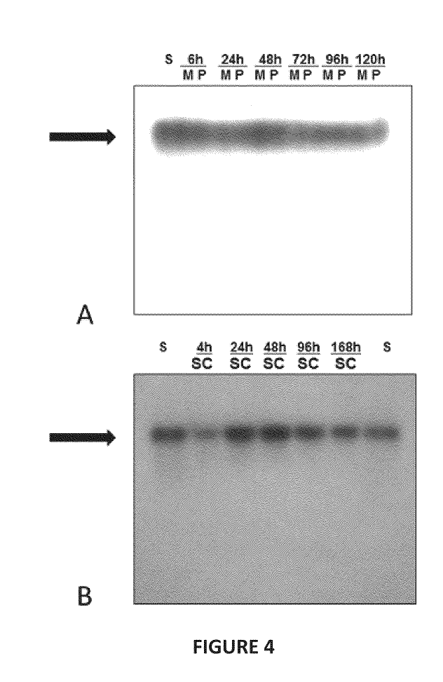

Heparin and heparan sulfate, for example, are degraded by a single enzyme known as heparanase. Experimental challenge of heparosan and N-sulfo-heparosan with heparanase, however, shows that since these polymers lack the O-sulfation of heparin and heparan sulfate, heparosan and N-sulfo-heparosan are not sensitive to enzymatic action in vitro by heparanase. These findings indicate that heparosan is not fragmented enzymatically in the extracellular compartments of the body, thereby indicating that heparosan is a stable material for use as a drug carrier. Experimentally, this stability is depicted in FIG. 4.

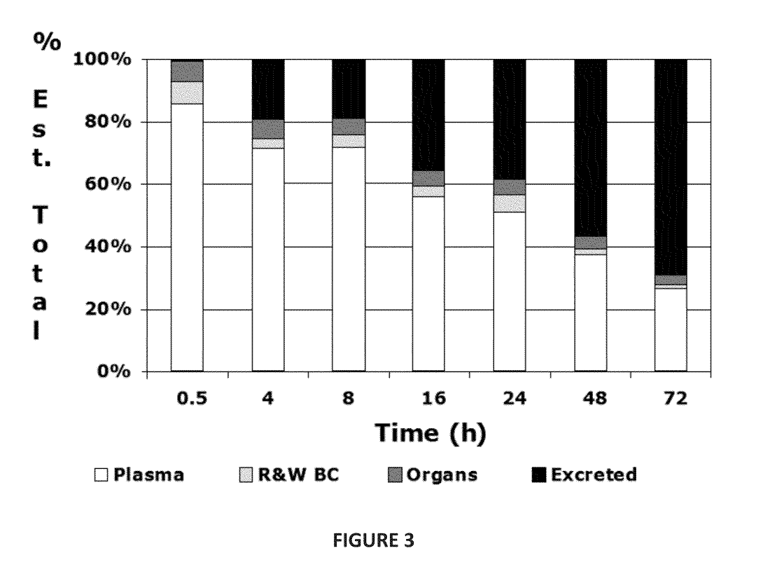

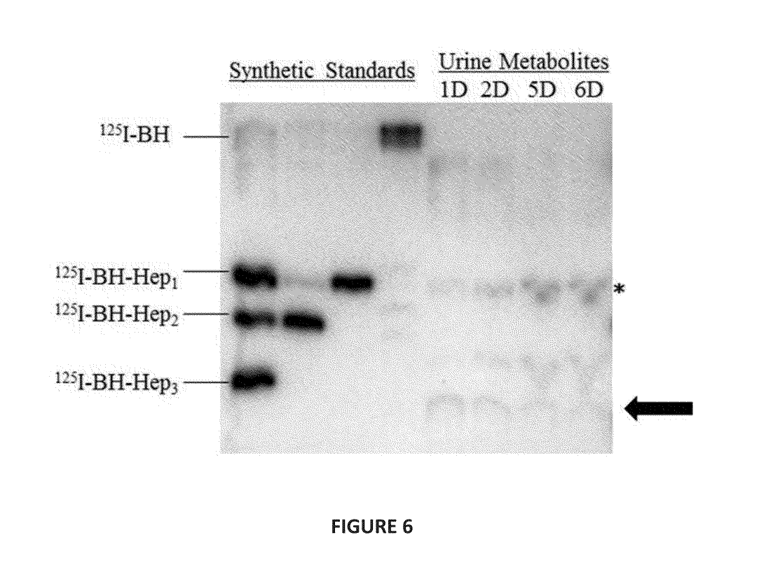

However, if heparosan or any of its fragments (e,g., generated by reactive oxygen species, etc.) is internalized into the lysosome, then the molecules will be degraded by resident beta-glucosidase and alpha-hexosaminidase enzymes (which remove one sugar at a time from the non-reducing termini of the GAG chain), similar to the degradation of heparin or hyaluronic acid. Therefore, the heparosan polymer is biodegradable and will not permanently reside in the body and thereby cause a lysosomal storage problem. A key advantage for therapeutic modification by complexation with heparosan polymer is that normal monosaccharides, GlcNAc and GlcUA, are the products of the eventual degradation. Experimentally, this biodegradation and excretion is depicted in FIGS. 3 and 6,

In contrast, some plastic-like carriers such as polyethylene glycol (PEG) or polymethacrylate have issues due to their synthetic, non-mammalian nature. PEG can degrade into reactive artificial aldehydes and ketones, which are toxic above certain levels. PEG and most other plastic-like carriers also tend to accumulate in the body, especially when present or utilized as one or more high molecular weight polymers. For treatments involving repeated dosing and/or high dose regimens, the accumulation of the carrier polymer in the patient's body or tissues is not desirable.

The normal roles of heparin/heparan sulfate in vertebrates include binding coagulation factors (inhibiting blood clotting) and growth factors (signaling cells to proliferate or differentiate). The key structures of heparin/heparan sulfate that are recognized by these factors include a variety of O-sulfation patterns and the presence of iduronic acid [IdoUA]; in general, polymers without these modifications do not alter clotting or cell growth. Heparosan-based materials which do not have such O-sulfation patterns, therefore, do not provoke unwanted clotting or cellular growth/modulation. As such, drug-heparosan complexes do not initiate clotting and/or cell growth processes and remain solely bio-reactive as per the drug or cargo constituent; heparosan is thus termed or deemed to be relatively biologically inert.

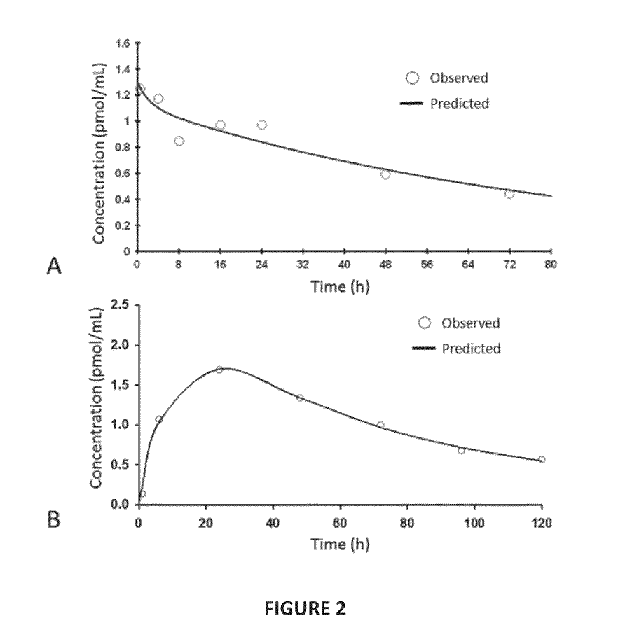

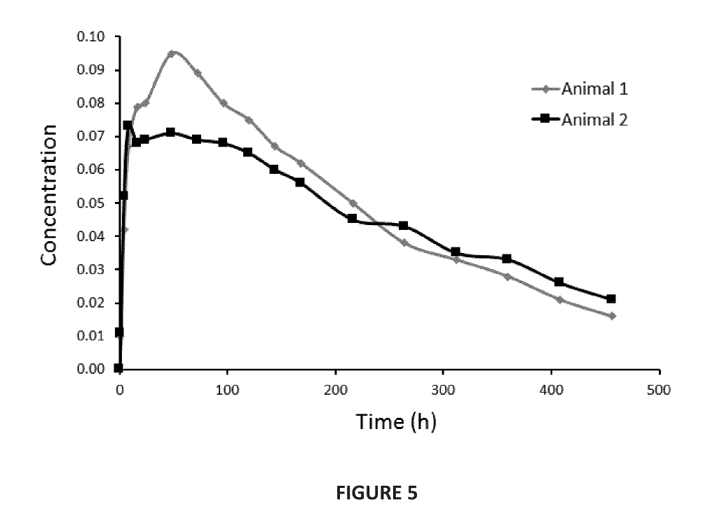

Hyaluronan (HA) has been used as a drug carrier, but it has a short half-life in the healthy animal due mainly to rapid uptake and clearance by a major receptor protein, HARE (HA Receptor for Endocytosis). While CD44, another HA receptor, is upregulated or more active in cancer cells compared to normal cells, the concurrent liver clearance mediated by HARE will also cause off-target localization of the drug, thus sickening or killing normal cells, which could result in toxic side effects. In addition, if some drug carried on the HA polymer is going to healthy tissue, then some portion of the injected drug is not fighting the cancer cells in the tumor and thus wasted. Heparosan, on the other hand, is not bound by HARE and thus will avoid these issues of off-target localization and reduced dosing effects. Experimentally, this long half-life is depicted in FIGS. 2 and 5.

Foreign or unnatural molecules stimulate the immune system. Heparosan polymer exists transiently during heparan sulfate and heparin biosynthesis in the Golgi apparatus as well as being found in unmodified polymer segments within mature heparan sulfate or heparin chains. In the latter case, the N- and O-sulfation reactions are not complete in mammals, so traces of the original heparosan remain; for example, approximately 1-5 unsulfated disaccharide repeats can he interspersed within the sulfated regions. Therefore, the body treats heparosan as ‘self,’ and does not mount an immune response. P. multocida Type D and E. coli K5 utilize heparosan coatings (called capsules) to ward off host defenses by acting as molecular camouflage. lndeed, scientists had to resort to using capsule-specific phages or selective GAG-degrading enzymes to type these heparosan-coated microbes, since a conventional antibody or serum could not be generated; heparosan is thus termed or deemed non-immunogenic or non-antigenic. A previously proposed protein-based carrier, polyglutamic acid, is not a naturally occurring sequence in the mammalian proteome, and thus could be the subject of an immune attack; this effect could be amplified by the typical repeated dosing regimens employed in cancer chemotherapy, whereby the boosting effect drives a weak antibody response to a higher level effect that could be problematic. The immune response to the foreign, non-self polyglutamic acid or an artificial, non-human (e.g., plastic-like) polymer could result in either antibody-mediated clearance of the drug (thus no or lower effectiveness and/or lead to an allergic response that may escalate to anaphylaxis (which can result in death in severe cases).

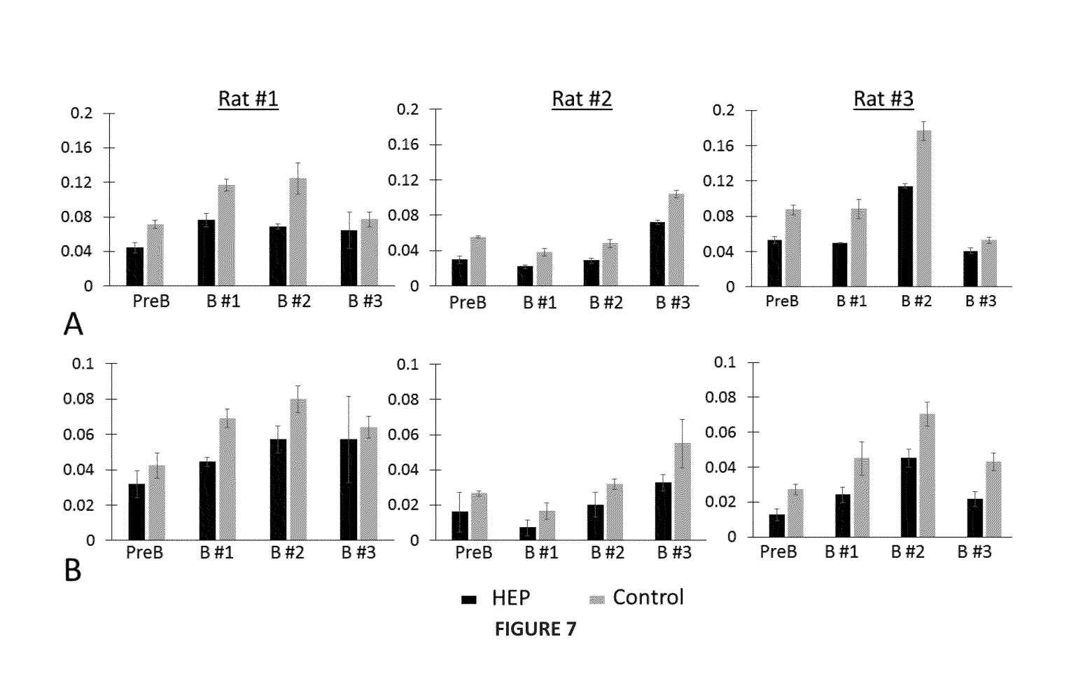

Heparosan, however, appears to be non-immunogenic based on rat tests where various heparosan-conjugates were repeatedly boosted and the subsequent sera tested for IgM or IgG (two main types of antibody molecules) via ELISA immunoassays. There was no signal when comparing wells coated with heparosan-BSA (bovine serum albumin) versus the background BSA-alone-coated wells, as depicted in FIG. 7.

Heparosan is a sugar polymer of the formula -[GlcNAc-alpha4-GlcUA-beta4]ₙ- where n is from 2 to about 5,000. The term “oligosaccharide” generally denotes n being from about 1 to about 11, while the term “polysaccharide” generally denotes n being equal to or greater than 12.

The term “complex” as used herein refers to a combination of two or more compounds formed by and held together through (1) coordination bonds, where one electron-deficient species accepts electrons from an electron-rich species (ligand), or (2) electrostatic interactions, where oppositely charged ion species interact favorably.

The term “cargo” as used herein refers to a drug, therapeutic, or other biologically active component present in a complex, while the term “vehicle” as used herein refers to the carrier of the cargo (e.g., the heparosan polymer) in the complex.

As used herein, the terms “active agent(s),” “active ingredient(s),” “pharmaceutical ingredient(s),” “therapeutic,” “medicant,” “medicine,” “biologically active compound,” and “bioactive agent(s)” are defined as drugs and/or pharmaceutically active ingredients. The presently disclosed and/or claimed inventive concept(s) may be used to encapsulate, attach, or bind drugs that are utilized as the pharmaceutically active agent in a composition, and/or the presently disclosed and/or claimed inventive concept(s) may he used to affect the storage, stability, longevity and/or release of said drugs. Non-limiting examples of the bioactive agents or anti-cancer agents include cisplatin, picoplatin, other platinum derivatives, and the like, as well as combinations thereof.

Many compounds have therapeutic potential for treating diseases such as cancer, but often have physical, chemical, and/or biological issues that limit their efficacy and/or cause toxicity. The presently disclosed and/or claimed inventive concept(s) describes the use of complexes of drugs and potential drug candidates with heparosan ([-4-GlcuA-1-beta-4-GIcNAc-1-alpha-4])ₙ), a naturally occurring, non-toxic sugar polymer, to increase therapeutic efficacy and/or limit side effects. These complexes provide methods for delivering such compounds that have benefits in comparison to other non-ideal strategies; these benefits include, but are not limited to: (i) slow administration of a single dose over a long period, (ii) repeated administration of smaller doses over an extended period, and/or (i)i) the use of synthetic or non-human polymers as delivery vehicles.

In one embodiment, heparosan forms a reversible coordination complex with certain metals, such as but not limited to, the transition metal platinum (Pt). They are also known as complex ions or coordination compounds because they are Lewis acid-base complexes. The ions or molecules that bind to metal ions to form these complexes are called ligands (from Latin, “to tie or bind”). These interactions are in general not thought to be mediated by conventional covalent bonds (i.e., sharing of electrons), but rather coordination bonds, where one electron-deficient species (metal) accepts electrons from an electron-rich species (ligand), For electrostatic complex interaction, the two or more oppositely charged ion species interact favorably, resulting in a hound state. For example, a positively charged (e.g., from primary, secondary, tertiary, or quaternary amine or nitrogen derivatives, cations, etc.) drug or drug candidate will interact with a negatively charged carboxylate group(s) on the heparosan polymer. If the therapeutic or therapeutic assembly has two or more positive charges, then the interaction would be stronger and/or last longer as a plurality of anionic carboxylates exist on any heparosan polymer chain and are available for complex formation.

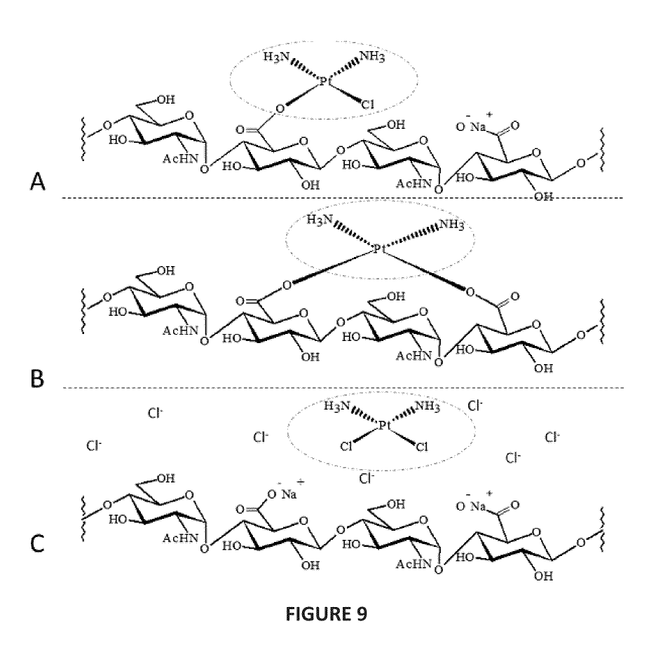

A more specific (but non-limiting) example of a drug that may be delivered in such a way with heparosan is cisplatin (Platinol, cisplatinum, or cis-diamminedichloroplatinum(ll)), a chemotherapy agent used to treat various cancers in mammals. During the synthesis of the heparosan/drug complexes, the chloride (Cl⁻) ligands on cisplatin are exchanged for a carboxylic acid (COO⁻) ligand of heparosan. FIG. 9 graphically depicts potential structures of cisplatin complexed with heparosan tetrasaccharide (wherein a partial structure of the longer heparosan polymer is represented). Similarly, picoplatin and other metal-containing compounds with similar ligand coordination abilities can interact in the same fashion.

In solutions without chloride ions, the prodrug is stable. After injection of the heparosan/drug complex (“HEPplex™”), the Cl⁻ions naturally present in the mammalian bloodstream (˜100 rnM) or other bodily fluids can re-exchange with the COO⁻ groups, thereby releasing the metal compound and thus forming exactly the same (or similar) drug or active pharmaceutical ingredient (API). The heparosan vehicle thus serves as a transient carrier for cisplatin, picoplatin, or a prodrug. In this embodiment, various size HEP polymers were complexed with the cisplatin in water. After complexation, the free drug was removed by ultrafiltration in water before testing.

Other aspects where heparosan may improve efficacy are by virtue of its molecular weight (MW) and its intrinsic behavior in the body (i.e., it is relatively inert and stable in extracellular spaces). Long chain heparosan polymers (>60-70 kDa or n>˜150-175) have long half-lives in the bloodstream and thus are able to prolong the action of small MW drugs or potential drug candidates that are complexed thereto when the drugs/drug candidates are normally excreted by the body via the renal filtration system. In addition, long heparosan polymers or particles (with an effective diameter of ˜20 to 200 nm) will be trapped in areas of tumors due to the malignancy's ‘leaky’ vasculature.

In addition to this lower molecular weight limit, if the polymer or particle is too large, then other systems in the normal tissues or organs (e.g., liver) will remove the particles. Thus, a window of from about 10-30 nm to about 100-200 nm is needed to target tumors. For example in Acharya & Sahoo (2011), it is stated that: “To take advantage of the EPR effect, it is critical for the nanocarriers to evade immune surveillance and circulate for a prolonged period. On the other hand, the kidneys are capable of filtering particles smaller than 10 nm and the liver can capture particles larger than 100 nm. Therefore, the ideal nanocarrier size is somewhere between 10 and 100 nm.” Similarly, it was noted that: “To produce long-circulating nanoparticles that can accumulate inside tumor tissues a diameter between 30 nm and 200 nm is desired” (Albanese et al., 2012). Another report states that: “EPR effect is mainly the function of molecular weight with molecules ranging 40-800 kDa can exhibit preferential tumor targeting” (Grobmeyer & Moudgil, 2010). The broad, semi-overlapping ranges reported in these studies are needed to describe different classes of molecules or nanoparticles with distinct geometries and behaviors (e.g., globular versus linear, flexible versus rigid, etc.) as well as due to the limitations and caveats of the particular size determination methods employed (e.g., size exclusion or gel permeation chromatography, electrophoresis, light scattering, viscosity, etc.). The heparosan polymers utilized in accordance with the presently disclosed and/or claimed inventive concept(s) can be produced in the optimal EPR ‘tumor targeting’ size ranges and molecular weights.

Bioactive Delivery of the Heparosan Complex

The heparosan complex may be administered, for example but not by way of limitation, parenterally, intraperitoneally, intraspinally, intravenously, intramuscularly, intravaginally, subcutaneously, intranasally, rectally, intraocularly, and/or intracerebrally. Dispersions of the heparosan complex may be prepared in, for example but not by way of limitation, glycerol, dextrose solutions, liquid poly[ethylene glycols], and mixtures thereof, as well as in oils. Under ordinary conditions of storage and use, such preparations of the heparosan complex may also contain a preservative to prevent the growth of microorganisms.

Pharmaceutical compositions suitable for injection use include (but are not limited to sterile aqueous solutions (where water soluble) or dispersions and sterile powders for the extemporaneous preparation of sterile injectable solutions or dispersions. In all cases, the composition must be sterile and must be fluid to the extent that easy syringeability exists. It must be stable under the conditions of manufacture and storage, and it must also be preserved against the contaminating action of microorganisms such as bacteria and fungi. The heparosan complex may be used in conjunction with a solvent or dispersion medium containing, for example, hut not by way of limitation, water, dextrose solutions, ethanol, a polyol (for example, glycerol, propylene glycol, liquid poly[ethylene glycol], and the like), suitable mixtures thereof, vegetable oils, and combinations thereof.

The proper fluidity of the heparosan complex may be maintained, for example, by the use of a coating (such as, but not limited to, lecithin), by the maintenance of the required particle size in the case of dispersion, and/or by the use of surfactants. Prevention of the action of microorganisms may be achieved by various antibacterial and antifungal agents, such as (but not limited to) parabens, chlorobutanol, phenol, ascorbic acid, thimerosal, and the like. In many cases, it may be desirable to include isotonic agents, such as (but not limited to) sugars or polyalcohols (such as mannitol and sorbitol), in the composition. Prolonged absorption of the injectable compositions may be brought about by including in the composition an agent that delays absorption, such as (but not limited to) aluminum monostearate or gelatin. However, due to the nature of platinum compounds like cisplatin, chloride-containing buffers (e.g., 0.9% isotonic saline, PBS, etc.) are not highly recommended solutions, as the free chloride ions will start to reverse the coordination complex during storage or before injection into the patient, thus partially negating its therapeutic action and potency.

Sterile injectable solutions may be prepared by incorporating a required amount of the heparosan-cargo complex in an appropriate solvent with one or a combination of ingredients enumerated above, as required, followed by filtered sterilization. Generally, dispersions are prepared by incorporating the heparosan-cargo complex into a sterile carrier that contains a basic dispersion medium and the required other ingredients from those enumerated above. In the case of sterile powders for the preparation of sterile injectable solutions, the methods of preparation may include vacuum drying, spray drying, spray freezing and freeze-drying that yields a powder of the active ingredient (i.e., the heparosan complex) plus any additional desired ingredient(s)from a previously sterile-filtered solution thereof.

It is especially advantageous to formulate parenteral compositions in dosage unit form for ease of administration and uniformity of dosage. The term “dosage unit form” as used herein refers to physically discrete units suited as unitary dosages for the subjects to be treated; each unit contains a predetermined quantity of heparosan-cargo complex calculated to produce the desired therapeutic effect. The specification for the dosage unit forms of the presently disclosed and/or claimed inventive concept(s) are dictated by and directly dependent on (a) the unique characteristics of the heparosan-cargo complex arid the particular therapeutic effect to be achieved, and (b) the limitations inherent in the art of compounding such a therapeutic compound for the treatment of a selected condition in a subject.

Aqueous compositions of the presently disclosed and/or claimed inventive concept(s) comprise an effective amount of the nanoparticle, nanofibril, or nanoshell, or chemical composition of the presently disclosed and/or claimed inventive concept(s) dissolved and/or dispersed in a pharmaceutically acceptable carrier and/or aqueous medium. The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous composition that contains an effective amount of the nanoshell composition as an active component and/or ingredient will be known to those of skill in the art in light of the present disclosure. Typically, such compositions may be prepared as injectables, either as liquid solutions and/or suspensions; solid forms suitable for use in preparing solutions and/or suspensions upon the addition of a liquid prior to injection may also be prepared; and/or the preparations may also be emulsified. Also, the heparosan vehicle can be used to enhance a secondary vehicle (e.g., liposomes, nanoparticles, etc.) that acts as a carrier or adjuvant for a drug.

Certain embodiments of the presently disclosed and/or claimed inventive concept(s) are directed to a composition that includes a drug complexed with a heparosan polymer to form a heparosan polymer-drug complex, wherein the drug-heparosan complex is formed through coordination bonds and/or electrostatic interactions.

The heparosan polymer present in the composition is biocompatible with a mammalian patient. Any heparosan polymers described or otherwise contemplated herein may be utilized as the heparosan polymer of the composition. For example, but not by way of limitation, the heparosan polymer may have a mass in a range of from about 500 Da to about 1,500 kDa. Particular non-limiting examples of ranges include, but are not limited to: ranges having a lower limit of from about 500 Da, about 600 Da, about 700 Da, about 800 Da, about 900 Da, about 1 kDa, about 2 kDa, about 3 kDa, about 4 kDa, about 5 kDa about 6 kDa, about 7 kDa about 8 kDa, about 9 kDa, about 10 kDa, about 11 kDa, about 12 kDa about 13 kDa about 14 kDa, about 15 kDa, about 16 kDa, about 17 kDa, about 18 kDa about 19 kDa, about 20 kDa, about 21 kDa about 22 kDa about 23 kDa, about 24 kDa, about 25 kDa, about 26 kDa, about 27 kDa, about 28 kDa, about 29 kDa, about 30 kDa, about 40 kDa and about 50 kDa; and an upper limit of from about 100 kDa, about 200 kDa, about 300 kDa, about 400 kDa, about 500 kDa, about 600 kDa, about 700 kDa, about 800 kDa, about 900 kDa, about 1,000 kDa, about 1,100 kDa, about 1,200 kDa, about 1,300 kDa, about 1,400 kDa, and about 1,500 kDa. Additional particular non-limiting range examples include a range of from about 600 Da to about 800 kDa, and a range of from about 27 kDa to about 1,300 kDa. The heparosan polymer may be monodisperse or polydisperse, and the heparosan polymer may he unepimerized and/or unsulfated.

The drug present in the composition may be any drug described or otherwise contemplated herein. For example, but not by way of limitation, the drug may include a transition metal compound, such as a platinum or vanadium compound. In another non-limiting example, the drug may be cisplatin, picoplatin or a Pt(II) compound.

When compared to the drug alone, the composition of the presently disclosed and/or claimed inventive concept(s) may possess at least one of the following: (a) increased retention in blood circulation of the mammalian patent; (b) reduced occurrence of accumulation in healthy tissues of organs of the mammalian patient; and (c) increased occurrence of accumulation in tumors or malignant growths of the mammalian patient.

In a particular non-limiting embodiment, the composition may include at least a first complex of a heparosan polymer with a first drug and a second complex of a heparosan polymer with a second drug. Alternatively, the same heparosan polymer chain may be complexed to two or more drug types,

Other embodiments of the presently disclosed and/or claimed inventive concept(s) are directed to a method of forming any of the compositions described or otherwise contemplated herein. In the method, a drug is complexed with a heparosan polymer to provide a composition comprising a heparosan polymer-drug complex as described or otherwise contemplated herein.

Additional embodiments of the presently disclosed and/or claimed inventive concept(s) are directed to a method in which a therapeutically effective amount of any of the compositions described or otherwise contemplated herein (i.e., a complex of a drug with a heparosan polymer) is administered to a mammalian patient so as to induce a therapeutic effect in the mammalian patient. In particular, non-limiting embodiments, the drug is released from the heparosan polymer in the mammalian patient.

Further embodiments of the presently disclosed and/or claimed inventive concept(s) are directed to a method for increasing the half-life of a drug in healthy tissues and increasing the targeting to diseased or tumorous tissues when administered to a mammalian patient. In the method, a drug is complexed with a heparosan polymer to provide a composition comprising any of the heparosan polymer-drug complexes described or otherwise contemplated herein. An effective amount of the composition is then administered to a mammalian patient so as to induce a therapeutic effect in the mammalian patient.

### EXAMPLES

Examples are provided herein below. However, the presently disclosed and/or claimed inventive concept(s) is to be understood to not be limited in its application to the specific experimentation, results and laboratory procedures. Rather, the Examples are simply provided as one of various embodiments and are meant to be exemplary, not exhaustive.

### Example 1

### Production of Heparosan for use in Complexation

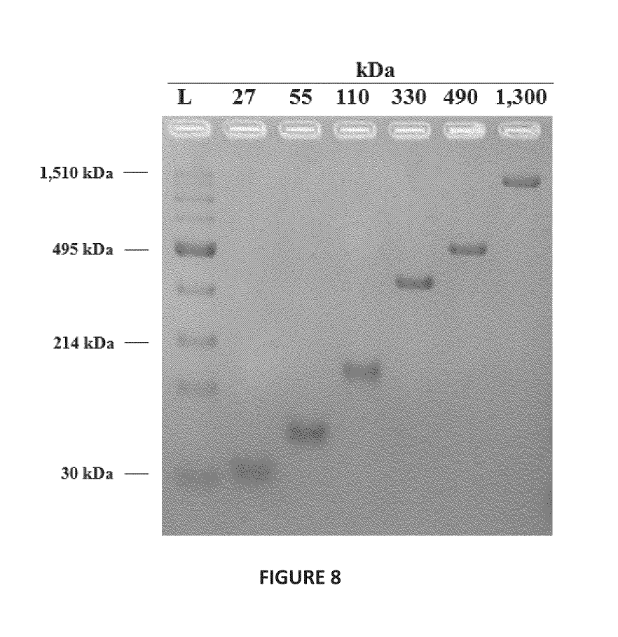

Defined GAG synthesis, and heparosan synthesis in particular, is rather versatile with respect to chemical functionality as well as size control. For example, US Patent Application Publication No. US 2008/0109236 (U.S. Ser. No. 11/906,704, filed Oct. 3, 2007) discloses a methodology for polymer grafting utilizing heparin/heparosan synthases from Pasteurella in order to provide heparosan polymers having a targeted size and that are substantially monodisperse at the desired size ranges (FIGS. 1 and 8). As such, the methodology of the '236 application can be applied to produce heparosan polymers suitable for complexation with a cargo molecule.

PmHS1 (see US Patent Application No. US 2010/0036001 for disclosure of the amino acid and nucleotide sequences thereof) was expressed as a carboxyl terminal fusion to maltose binding protein (MBP) using the pMAL-c2X vector (New England BioLabs, Ipswich, Mass.). To facilitate extracting the enzymes, the expression host E. coli XJa (Zymo Research, Irvine, Calif.), which encodes a phage lysin enzyme, was employed and used for simple freeze/thaw lysis. Cultures were grown in Superior Broth (AthenaES, Halethorpe, Md.) at 30° C. with ampicillin (100 μg/mI) and L-arabinose (3.25 mM). At mid-log phase, isopropyl β-D-1-thiogalactopyranoside (IPTG) (0.2 mM final) was added to induce fusion protein production. One hour after induction, the cultures were supplemented with fructose (12.8 mM final) and grown for approximately 5-12 hours before harvesting by centrifugation at 4° C. The bacteria were resuspended in 20 mM Tris, pH 7.2, with protease inhibitor cocktail on ice, then frozen and thawed twice, thus allowing lysin to degrade the cell walls. The lysates were clarified by centrifugation.

The synthase was affinity purified via the fused MBP unit using amylose resin (New England BioLabs Ipswich, Mass.). After washing extensively with column buffer (20 mM Tris, pH 7.2, 200 mM NaCl, 1 mM EDTA), the protein was eluted in column buffer containing 10 mM maltose. Protein concentration was quantitated by the Bradford assay (Pierce, Rockford, Ill.) using a bovine albumin serum standard. The purification was monitored by SDS-PAGE with copper negative staining (which adds comparable sensitivity as conventional silver staining) followed by Coomassie blue staining. The enzyme (approximately 90-95% pure; yield ˜10 mg per liter of culture) can be used directly after buffer exchange into 50 mM Tris, pH 7.2, by ultrafiltration. Further purification by anion-exchange chromatography provided an approximately 95-99% pure PmHS1 enzyme.

A heparosan polysaccharide (having a molecular weight of approximately 200-300 kDa) derived from the spent fermentation broth of P. multocida Type D cultures was converted into heparosan tetrasaccharide (4-mer, having a molecular weight of approximately 700 Da), the starting material for the primers described later herein. P. multocida Type D cells were grown in a proprietary synthetic media at 37° C. in shake flasks for approximately 24 hrs. Spent culture medium (the liquid part of culture after microbial cells were removed) was harvested (by centrifugation at 10,000×g, 20 min) and deproteinized (solvent extraction with chloroform). The very large anionic heparosan polymer (“fermentation heparosan,” having a molecular weight of approximately 200-300 kDa) was isolated via ultrafiltration (30 kDa molecular weight cut-off Amicon ultrafiltration filter unit, EMD Millipore, Billerica, Mass.) and ion exchange chromatography (NaCl gradient on Q-Sepharose; Pharmacia, Stockholm, SE). Heparosan from E. coli K5 cultures can also be used, but the polymers produced therefrom typically have an initially lower molecular weight than the heparosan polymers produced by P. multocida Type D.

Heparosan oligosaccharides ((GlcUA-GlcNAc)ₙ-(GlcUA-anhydromannitol), n=1, 2 or 3) were prepared by partial deacetylation of heparosan polysaccharide with base, nitrous acid hydrolysis, and reduction; these polymers contain intact non-reducing termini but an anhydromannitol group at the reducing end. The fragments were purified by gel filtration on a P2 column (BioRad, Hercules, Calif.) in 0.2 M ammonium formate, followed by normal phase thin layer chromatography (TLC) on Whatman silica plates (GE Healthcare Bio-Sciences, Pittsburgh, Pa.) with n-butanol/acetic acid/water (1:1:1). The bands were detected by staining of side lanes with napthoresorcinol. The size and purity of oligosaccharides were verified by matrix assisted laser desorption ionization time of flight mass spectrometry (MALDI-ToF MS). Alternatively, acid hydrolysis or enzymatic cleavage of HEP polymer yields oligosaccharides that can also be employed for use. Alternatively, the acceptor may be a synthetic glycoside or heparosan fragment. The source of the acceptor is not the limiting factor here; rather, it is the acceptor's ability to be elongated by a heparosan synthase and to synchronize the reaction is most relevant.

Synchronized polymerization reactions were used to produce monodisperse polymers as previously described and disclosed in the '236 patent application publication. The formation of heparosan with a narrow size distribution (i.e., monodisperse) is dependent on the ability of the PmHS1 enzyme to be primed by acceptors (thus avoiding a slow de novo initiation event yielding out of step elongation events) and efficiently transfer monosaccharides from UDP-sugars. Recombinant PmHS1 synthesizes heparosan chains in vitro if supplied with both required UDP-sugars according to the equation:

nUDP-GlcUA+n UDP-GlcNAc→2n UDP+[GlcUA-GlcNAc]ₙ

However, if a heparosan-like oligosaccharide ([GlcUA-GlcNAc]ₓ or similar variations) is also supplied in vitro, then the overall incorporation rate is elevated up to approximately 25-fold. The rate of initiation of a new chain de novo is slower than the subsequent elongation (i.e., repetitive addition of sugars to a nascent HA molecule). The observed stimulation of synthesis by exogenous acceptor primer appears to operate by bypassing the kinetically slower initiation step, allowing the elongation reaction to predominate as in the following equation:

n UDP-GlcUA+n UDP-GlcNAc+z[GlcUA-GlcNAc]ₓ→2n UDP+z[GlcUA-GlcNAc]ₓ₊ₙ

If there are many termini (i.e., z is large, meaning many acceptor molecules are present), then a limited amount of UDP-sugars will be distributed among many molecules and thus result in many short polymer chain extensions. Conversely, if there are few termini (i.e., z is small, and thus few acceptor molecules are present), then the limited amount of UDP-sugars will be distributed among few molecules and thus result in long polymer chain extensions. Thus, by controlling the molar ratio of acceptor to UDP-sugar, it is possible to select the final polymer size desired, as shown in FIG. 8. Typically, from about 50% to about 90% of the starting UDP-sugars were consumed in the reactions on the basis of polysaccharide recovery.

Alternatively, if size control is not as critical, then “fermentation heparosan” or its fragments (generated by acid, base, enzyme, or physical cleavage methods known to those of skill in the art) will suffice as the vehicle (FIG. 1). For example, spent cultures of heparosan-producing microbes such as Pasteurella multocida Type D, Escherichia coli K5, Avibacterium species, and any recombinant system using heparosan biosynthesis genes from such organisms (providing the molecular weight of the polymer product is sufficient), may be used as the starting material for heparosan employed in the presently disclosed and/or claimed inventive concept(s). Similarly, chemically manufactured heparosan may be utilized. As will be appreciated by one of ordinary skill in the art, therefore, it is not the source or manner in which the heparosan is made that is controlling; rather, it is the particular use to which the heparosan will be put. If size is critical, recombinant chemoenzymatic production is more desirable. In situations where size control is of a secondary or lesser importance, fermentation heparosan (or its derivatives) may be used. As such, the use of heparosan from any source or produced by any methodology is intended to be within the presently disclosed and/or claimed inventive concept(s). Likewise, it is not the nature or manner of the complexation between heparosan and the drug that is controlling; rather, it is the particular use to which the heparosan will be put.

The yield and molecular weight size distribution of the heparosan was checked by (a) carbazole assays for uronic acid, and (b) agarose gel electrophoresis (1× TAE buffer, 0.8-1.5% agarose) followed by Stains-AH detection. The carbazole assay is a spectrophotometric chemical assay that measures the amount of uronic acid in the sample via production of a pink color; every other sugar in the heparosan chain is a glucuronic acid (GlcUA). The heparosan polymer size was determined by comparison to monodisperse HA size standards (HA Lo-Ladder, Hyalose, LLC, Oklahoma City, Okla.) run on gels. The detection limit of the carbazole and the gel assays is approximately 5-15 micrograms of polymer. Any endotoxin was removed by passage through an immobilized polymyxin column (Pierce, Rockford, Ill.); the material is then tested with a Limulus amoebacyte-based assay (Cambrex, East Rutherford, N.J.) to assure that the heparosan contains <0.05 endotoxin units/mg solid (based on USP guidelines),

Examples of the productions of monodisperse heparosan are shown in FIG. 8 where providing various levels of primer yielded different M_{w} (weight average molecular mass) products with low polydispersity (M_{w}/Mₙ; Mₙ=number average molecular weight). For reference, the polydispersity value for an ideal monodisperse polymer equals 1. In contrast, reaction without an acceptor resulted in a large product that was significantly polydisperse i.e., it contains heparosan polymers of varying size and length.

The polymerization by synthases in the presence of an acceptor is a synchronized process. Reactions without acceptor exhibit a lag period interspersed with numerous, out of step initiation events that yield a short heparosan oligosaccharide. Once any chain is formed, the heparosan polymer is elongated rapidly. Other new chains that arise later during the lag period are also elongated rapidly, but the size of these younger chains never catches up to the older chains in a reaction with a finite amount of UDP-sugars. In contrast, in reactions containing an acceptor, all heparosan chains are elongated in parallel in a nonprocessive fashion, resulting in a more homogenous final polymer population. However, as noted before, heparosan produced chemoenzymatically either without acceptor or with acceptor falls within the scope of the presently disclosed and/or claimed inventive concept(s).

The enzymological properties of recombinant PmHS1 described above also allow for the control of heparosan polymer size in chemoenzymatic syntheses. First, as noted above, the rate-limiting step in vitro appears to be the chain initiation step. Therefore, PmHS1 transfers monosaccharides onto the existing heparosan acceptor chains before substantial de novo synthesis. Second, the enzyme polymerizes heparosan in a rapid nonprocessive fashion in vitro. Therefore, the amount of primer should affect the final size of the product when a finite amount of UDP-sugar is present. The synthase adds all available UDP-sugar precursors to the nonreducing termini of acceptors as in the equation:

n UDP-GlcUA+n UDP-GlcNAc+z[GlcUA-GlcNAc]ₓ→2n UDP+z[GlcUA-GlcNAc]_{x+(n/z)}

Thus, by controlling the molar ratio of acceptor to UDP-sugar, it is now possible to select the final heparosan polymer size desired. Typically, from about 50% to about 90% of the starting UDP-sugars are consumed in the reactions on the basis of polysaccharide recovery.

The size distribution and the hydrodynamic size of the heparosan polymers produced was determined by high performance size exclusion chromatography-multi angle laser light scattering (SEC-MALLS). Polymers (2.5 to 12 μg mass; 50 μl injection) were separated on PL aquagel-OH 30 (8 μm), -OH 40, -OH 50, -OH 60 (15 μm) columns (7.5×300 mm, Polymer Laboratories/Varian Medical Systems, Inc., Palo Alto, Calif.) in tandem or alone, as required by the size range of the polymers to be analyzed. The columns were eluted with 0.2 M sodium nitrate (to preserve heparosan/cisplatin complexes that are sensitive to chloride found in PBS) at 0.5 ml/min. MALLS analysis of the eluant was performed by a DAWN DSP Laser Photometer in series with an OPTILAB DSP Interferometric Refractometer (632.8 nm; Wyatt Technology, Santa Barbara, Calif.). The ASTRA software package was used to determine the absolute average molecular mass using a dnidc coefficient of 0.153 determined for HA, a polymer with the exact same sugar composition as heparosan, by Wyatt Technology.

MALLS analyses of various heparosan preparations are shown in Table 2. The hydrodynamic sizes observed indicate that molecules with the appropriate size for the EPR effect (˜10-100 nm or ˜30-200 nm as described in the literature) were made. Likewise, in another indication of size, an average molecular weight in the range of ˜40-800 kDa was achieved with heparosan preparations. In summary, both fermented heparosan from P. multocida in vivo, as well as heparosan produced enzymatically in vitro, can serve as the carrier for drug; the source is not critical as long as the MW is sufficient.

TABLE 2
Molecular Weight & Hydrodynamic
Size of Heparosan Polymers by MALLS-SEC

| Heparosan | Average Molecular | Average Hydrodynamic |
|---|---|---|
| Batch | Weight, M_{w} (kDa) | Diameter (nm) |
| 1 | 45.1 | 34 |
| 2 | 743 | 44 |
| 3 | 95.2 | 52 |
| 4 | 144 | 64 |
| 5 | 296 | 106 |

### Example 2

### Synthesis of Heparosan/Therapeutic Compound Coordination Complexes

In this example of the synthesis of heparosan-cisplatin complexes, 10 mg of 300-kDa heparosan (average MW=296 kDa; 32.5 nmoles; Batch #5 in Table 2) in 1 mL of water in a 2-mL screw cap tube was combined with either 1.5 mg of cisplatin (5 μmoles; Sample A) or with 3 mg of cisplatin (10 μmoles; Sample B), which was then filled with inert gas (argon) to reduce oxidation of cisplatin. However, the drug loading on the polymer's carboxyl groups can be targeted from low loading to the highest possible (i.e., where all carboxyl groups are complexed with the platinum compound) by altering the ratio of drug to polymer in the reaction. Likewise, various size HEP polymers can be employed as the starting material; if the polymer falls within the EPR targeting size range, then the polymer will be trapped in the tumor. The tube was then mixed by slow rotation in the dark for 72 hours at room temperature. However, these reaction conditions are to be understood to simply be one non-limiting example of reaction conditions; one of ordinary skill in the art will understand that the exact parameters of concentration, solvent, mixing, temperature, time, inert gas, etc., can be varied and the reaction still proceed; therefore, various parameters of concentration, solvent, mixing, temperature, time, etc. that are discernible to a person having ordinary skill in the art also fall within the scope of the presently disclosed and/or claimed inventive concept(s). Similarly, other complex-forming drugs (e.g., picoplatin) could be employed. The purification of heparosan-cisplatin may be accomplished by ultrafiltration, dialysis, or size exclusion chromatography. Here, the reaction mixture was diluted 14-fold with water, mixed, and then spun in a 10-kDa molecular weight (MW) cutoff Amicon ultrafiltration unit (EMD Millipore, Billerica, Mass.) at 3,000=g for 1 hour. This dilution and ultrafiltration process was repeated three more times to remove any uncomplexed, low MW cisplatin from the high MW heparosan/drug complexes. The material was then filter-sterilized through a 0.45 μm membrane and stored frozen. However, these purification conditions are to be understood to simply be one non-limiting example of purification conditions; one of ordinary skill in the art will understand that the exact parameters of filtration, chromatography, buffers, time, etc. can be varied and the purification still be effective; therefore, various additional parameters of filtration, chromatography, buffers, time, etc. that are discernible to a person having ordinary skill in the art also fall within the scope of the presently disclosed and/or claimed inventive concept(s).

The amount of cisplatin complexed to heparosan was measured by two assays: (i) a spectrophotometric assay with O-phenylenediamine [OPD] (Basotra et al, 2013) or (ii) Inductively coupled plasma mass spectrometry [ICP-MS] (EPA Methods 6020A, 3005, and 3010).

In brief, for O-phenylenediamine testing, a sample of up to 400 μl was adjusted with 153 μl 0.6M sodium phosphate buffer (pH 6.8) and mixed; then 47 μl of 30 mg/mL O-phenylenediamine was added. The assay reaction was heated at 100° C.×10 min and cooled to room temperature before adding 1.4 mL dimethylformamide, mixing, and reading the absorbance at 706 nanometers (nm). This quick assay is convenient for semi-quantitative assessment of the cisplatin in a sample,

In brief, for ICP-MS testing, acid was added to the sample, heated on a hot plate (all of the metals dissolve and disassociate in solution). Deionized water was added to make up for the volume lost and the sample was then analyzed on the ICP-MS instrument (Agilent 7900 ICP-MS, Agilent Technologies, Santa Clara, Calif.). This assay allowed quantitative assessment of the total platinum (Pt) in a sample, but requires specialized equipment and trained operators.

The size of various heparosan (HEP) molecules (Table 2) as well as their cisplatin complexes was measured by MALLS-SEC (Table 3). The data show that the hydrodynamic size range for these polymeric carriers and their drug complexes are within the optimal tumor targeting size window described above. The data obtained in Table 3 indicates that in a complex, cisplatin adds to the molecular weight (MW) of the starting heparosan (i.e., HEP MW increases when drug is added in comparison to the starting sugar chains), but the complexes' hydrodynamic size decreases slightly (e.g., coordination as in FIG. 9, Panel B and/or loss of some negative self-repulsive groups of heparosan upon coordination causes HEP prodrug molecules to be more compact than HEP alone), Also, as shown in FIG. 8 agarose gel analyses indicated that the molecular weights of certain heparosan preparations used for cisplatin prodrug synthesis are suitable for tumor-targeting or the EPR effect. These are non-limiting examples of the types of complexing prodrugs that are possible; it is known in the art that the exact parameters of loading level (e.g., low, intermediate, or complete complexation), metal compounds (e.g., picoplatin, one or more drug compound types per chain, various metals, etc.), heparosan size (e.g., 40-800 kDa), etc., may be varied to optimize for the desired effect.

TABLE 3
Molecular Weight & Hydrodynamic Size of
Heparosan/Cisplatin Complexes

| | Drug Loading* | Average MW, | Average Hydrodynamic |
|---|---|---|---|
| Sample | (Pt/HEP w/w) | M_{w} (kDa) | Diameter (nm) |
| Starting HEP | 0 | 296 | 106 |
| A | 6.1% | 310 | 96.2 |
| B | 8.3% | 316 | 95.6 |

*measured by ICP-MS method

### Example 3

### Drug Release Studies from Heparosan/Platinum Drug Complexes and Uses Thereof

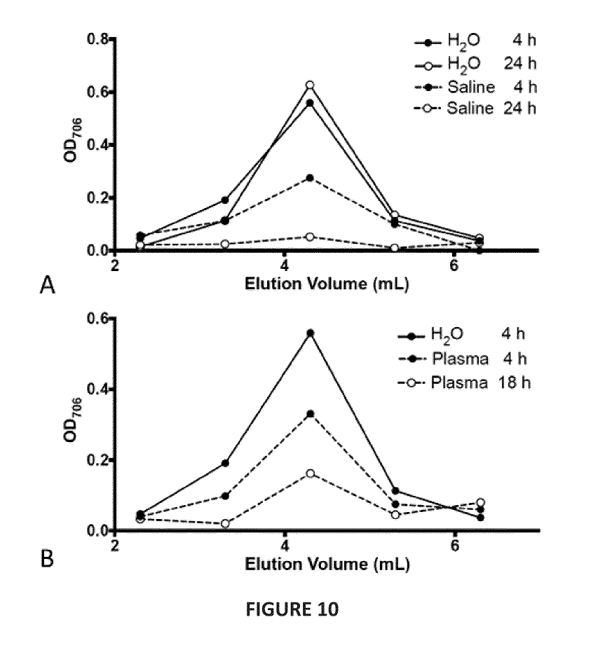

As described earlier, the heparosan/cisplatin complexes are formed in water and are stable, but once injected into the mammalian patient, the substantial amount of chloride ions (˜100 mM) will reverse the carboxylate/platinum complex (FIG. 9). The heparosan/drug complexes were tested for drug release under physiological conditions (0.1 M NaCl, 10 mM HEPES, pH 7.2 )or with vertebrate-derived body fluids (e.g., ultrafiltered chicken sera (Sigma Aldrich, St. Louis, Mo.) or human plasma (Innovative Research, Inc., Novi, Mich.)). In brief, the incubation mixtures were incubated at 37° C. for various times (0.1 to 24 hrs) and then analyzed by size exclusion chromatography (Sephadex G25-prepacked PD-10 columns, GE Healthcare Bio-Sciences, Pittsburgh, Pa.) eluted in 0.1 M Na phosphate, pH 7.2 (FIG. 10). The fractions were tested for sugar content (via carbazole assay) and cisplatin (via o-PD) and/or total platinum (via ICP-MS assays). Heparosan and its drug complexes eluted in the void volume, while the free drug eluted in the included volume. FIG. 10 shows some examples of the starting materials (here, 296 kDa polymer with Pt/HEP ratio of 8.3% w/w in water) and the challenged (NaCl or plasma samples. As predicted, the heparosan complexes release the free drug under conditions expected in the mammalian patient. In these examples, the approximate half-life for drug release in vertebrate body fluids was approximately 10 hours.

In summary, this transient pro-drug carrier strategy using heparosan complexes that do not alter the API structure (which are typically FDA-approved and their behavior is well known or characterized) when released into the body will reduce treatment time and side effects as well as increase efficacy of APIs or drug candidates.

To date, several synthetic polymers have been introduced into clinical practice, including polyethylene glycol (PEG), poly-styrene maleic anhydride copolymer (SMA), and N-(2-hydroxypropyl)-methacrylamide copolymer (HPMA). Reviews have documented the use of some of these polymers in cancer therapy (Fuertges et al. (1990); Maeda (1991); Putnam et al. (1995); Duncan et al. (1994)). Heparosan has benefits over these other synthetic polymers in that this human-like sugar polymer can be made in a wider variety of chain sizes with a narrow size distribution, is not immunogenic, and is metabolically degraded in cells via a pathway similar to heparin, a FDA-approved drug.

Heparosan has benefits over hyaluronan (HA) in that the former sugar is relatively inert (e.g., not rapidly cleared in bloodstream) and stable in extracellular spaces (e,g., not cut by heparanase), and therefore has a long half-life in the bloodstream; on the other hand, HA can be cleaved by enzymes and/or bound by receptors not present on cancer cells (i.e., non-target healthy cells will also bind and uptake HA), and HA also has a much shorter half-life in the bloodstream. Similarly, chondroitins and heparan sulfate/heparin are bioactive in the body and possess shorter half-lives in the bloodstream than heparosan.

Polyglutamic acid, a non-human polypeptide, has potential for immunogenicity, and its size control is not as flexible as the heparosan system.

### Example 4

### Drug Complexation to Heparosan Through Electrostatic Interactions

To produce electrostatic complexes, where the heparosan carboxylate groups will ionically bond with the positively charged moiety or moieties of the drug or drug candidate, several methods can be utilized. In one non-limiting example, ion exchange resins are utilized, where the cationic drug is loaded onto an anionic bead, a heparosan solution is contacted with the beads, and the complexes elute from the column, batch process, or similar elution method. In another non-limiting example, intermediary buffers are used, where acidic heparosan solutions are neutralized with the base form of the drug. In yet another non-limiting example, co-precipitation is utilized, where two appropriate pH solutions (one of the drug or drug candidate plus one of the heparosan) are combined and mixed to produce a complexed or particulate species. However, it is to be understood that the examples provided above are for purposes of illustration only; any similar methods that are discernible to a person having ordinary skill in the art also fall within the scope of the presently disclosed and/or claimed inventive concept(s).

Electrostatic complexes can be separated, isolated, and/or concentrated by any methods known in the art or otherwise contemplated herein. In one non-limiting example, ultrafiltration or tangential flow filtration against water or low ionic strength buffers is utilized to capture and concentrate high molecular weight complexes. In another non-limiting example, chromatography (e,g., employing gel filtration, solid-phase extraction, etc.) is used. In another non-limiting example, solvent-mediated precipitation (e.g., add alcohols, acetone, etc. to the heparosan/drug solution utilized in tandem with isolation methods, as in other non-limiting examples, centrifugation, flocculation, and/or filtration capture (e.g., to harvest particulate complexes). However, it is to be understood that the examples provided above are for purposes of illustration only; any similar methods that are discernible to a person having ordinary skill in the art also fall within the scope of the presently disclosed and/or claimed inventive concept(s).

Non-limiting examples of single amine-containing anti-cancer drugs and drug candidates that may be complexed with heparosan using this electrostatic complex methodology include, but are not limited to, adriamycin, doxirubicin, epirubicin, eribulin, discodermolide, combinations thereof, and the like. Non-limiting examples of multi-amine-containing anti-cancer drugs (which should form stronger complexes with heparosan) suitable for electrostatic complexation include, but are not limited to, vinflunine cevipabulin, omabraulin, indibulin, combinations thereof, and the like. Other cationic drugs with different medical applications, such as (but not limited to) the anti-parasitic drugs chloroquine and its analogs, are also amenable to this process.

In some cases, certain non-polar molecular structures of the drug or drug candidate, such as (but not limited to) hydrophobic aromatic rings or alkyl chains, may interact with the non-polar portions of an adjacent drug or drug candidate on the same or an adjacent heparosan polymer (e.g., stacking or aligning of multiple drug molecules along the heparosan backbone); alternatively and/or in addition thereto, these molecular structures may interact with the heparosan acetyl group or the non-polar C-H rich face of the sugar pyranose ring. These secondary hydrophobic interactions may enhance electrostatic complex stability and ease of processing during manufacture, and may also result in a longer half-life in the mammalian patient. Such complexes may also be less soluble in aqueous solutions and form nanoparticles that are also suitable for tumor trapping strategies.

Thus, in accordance with the presently disclosed and/or claimed inventive concept(s), there has been provided a complex wherein a heparosan molecule serves as the vehicle for carrying a cargo in a heparosan-cargo complex, as well as methods of producing and using same. Although the presently disclosed and/or claimed inventive concept(s) has been described in conjunction with the specific drawings and language set forth above, it is evident that many alternatives, modifications and variations will be apparent to those skilled in the art. Accordingly, it is intended to embrace all such alternatives, modifications, and variations that fall within the spirit and broad scope of the presently disclosed and/or claimed inventive concept(s).

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR §1.97 will be submitted separately.
Acharya, S., & Sahoo, S K. (2011) PLGA nanoparticles containing various anticancer agents and tumour delivery by EPR effect. Advanced Drug Delivery Reviews, 63 (3):170-83.
Albanese, A., Tang, P S., & Chan, W C W. (2012) The effect of nanoparticle size, shape, andsurface chemistry on biological systems. Annual Review of Biomedical Engineering, 14:1 -16.
Basotra, M., Singh, S K., & Gulatj, M. (2013) Development and Validation of a Simple and Sensitive Spectrometric Method for Estimation of Cisplatin Hydrochloride in Tablet Dosage Forms: Application to Dissolution Studies. ISRN Analytical Chemistry. 2013.
Cai, S., Xie, Y., Bagby, T R., Cohen, M S., & Forrest, M L. (2008) Intralymphatic chemotherapy using a hyaluronan-cisplatin conjugate. Journal of Surgical Research. 147(2):247-252.
DeAngelis, P L. (2002) Microbial glycosaminoglycan glycosyltransferases. Glycobiology. 12(1):9-6.
Duncan, R., & Spreafico, F. (1994) Polymer conjugates. Pharmacokinetic considerations for design and development. Clinical Pharmacokinetics. 27:290-306.
Fuertges, F., & Abuchowski, A. (1990) The clinical efficacy of poly(ethylene glycol)-modified proteins. Journal of Controlled Release, 11:139-148.
Gerlowski, L E. and Jain, R K. (1986) Microvascular permeability of normal and neoplastic tissues. Microvascular Research, 31(3):288-305.
Grobmeyer, S R, & Moudgil, B M. (2010) Cancer Nanotechnology: Methods and Protocols. (S. R. Grobrneyer & B. M. Moudgil, Eds.). New York, N.Y.: Humana Press.
Jain, R K. (1998) The next frontier of molecular medicine: Delivery of therapeutics. Nature Medicine, 4:655-657.
Jeong, Y I., Kim, S T., Jin, S G., Ryu, H H., Jin, Y H., Jung, T Y., Kim, I Y. & Jung, S. (2008) Cisplatin-incorporated hyaluronic acid nanoparticles based on ion-complex formation. Journal of Pharmaceutical Sciences. 97(3):1268-76.
Li, C. (2002) Poly(L-glutamic acid)-anticancer drug conjugates, Advanced Drug Delivery Reviews, 13; 54(5):695-713.
Maeda, H. and Matsumura, Y. (1989) Tumoritropic and lymphotropic principles of macromolecular drugs. Critical Reviews™ in Therapeutic Drug Carrier Systems, 6(3):193-210.
Maeda, H. (1991) SMANCS and polymer-conjugated macrorriolecular drugs: advantages in cancer chemotherapy. Advanced Drug Delivery Reviews, 6(2):181-202.
Maeda, M., Takasuka, N., Suga, T., Uehara N., & Hoshi, A. (1993) Antitumor activity of a new series of platinum complexes: trans(+/−)-1,2-cyclohexanediammineplatinum(II) conjugated to acid polysaccharides. Anticancer Drugs, 4(2):167-171.
Peng, X H., Wang, Y., Huang, D., Wang, Y., Shin, H J., Chen, Z., Spewak, M B., Mao, H., Wang, X., Wang, Y., Chen, Z., Nie, S., Shin, D M. (2011) Targeted Delivery of Cisplatin to Lung Cancer Using ScFvEGFR-Heparin-Cisplatin Nanoparticles. ACS Nano, 5 (12):9480-9493.
Putnam, D., & Kopecek, J. (1995) Polymer conjugates with anticancer activity. Advances in Polymer Science, 122:55-123.
Ringsdorf, H. (1975) Structure and properties of pharmacologically active polymers. Journal of Polymer Science: Polymer Symposia, 51:135-153.
Roberts, W G., & Palade, G E. (1997) Neovasculature induced by vascular endothelial growth factor is fenestrated. Cancer Research. 57:765-772.
Schechter, B., Pauzner, R., Amon, R., & Wilchek, M. (1986) Cis-platinum(II) complexes of carboxymethyl-dextran as potential antitumor agents. I. Preparation and characterization. Cancer biochemistry biophysics. 8(4):277-87.
Zhang, J S., Imai, T., & Otagiri, M. (2000) Effects of a cisplatin-chondroitin sulfate A complex in reducing the nephrotoxicity of cisplatin. Archives of Toxicology. 74(6),:300-7.
Zhang, J S., Imai, T., Suenaga, A. & Otagiri M. (2002) Molecular-weigh -dependent pharmacokinetics and cytotoxic properties of cisplatin complexes prepared with chondroitin sulfate A and C. International Journal of Pharmaceutics. 240(1-2):23-31.
US Patent Application Publication No. US 2013/0259944 A1, “Methods and compositions for treating cancer with platinum particles,” published Oct. 3, 2013, to Shin et al.
U.S. Pat. No. 8,088,412 B2, “Intralymphatic chemotherapy drug carriers,” issued Jan. 3, 2012, to Forrest et al.
U.S. Pat. No. 8,895,076 B2, “Liquid composition of cisplatin coordination compound,” issued Nov. 25, 2014, to Kataoka et al.

1. A composition, comprising:
a drug; and
a heparosan polymer, wherein the heparosan polymer is biocompatible with a mammalian patient; and
wherein the drug is complexed with the heparosan polymer to form a heparosan polymer-drug complex, wherein the drug-heparosan complex is formed through coordination bonds and/or electrostatic interactions.

2. The composition of claim 1, wherein the heparosan polymer has a mass in a range of from about 600 Da to 800 kDa.

3. The composition of claim 1, wherein the heparosan polymer has a mass in a range of from about 27 kDa to about 1,300 kDa.

4. The composition of claim 1, wherein the heparosan polymer is monodisperse.

5. The composition of claim 1, wherein the heparosan polymer is polydisperse.

6. The composition of claim 1, wherein the heparosan polymer is unepimerized.

7. The composition of claim 1, wherein the heparosan polymer is unsulfated.

8. The composition of claim 1, wherein the drug comprises a transition metal compound, and wherein the metal is platinum or vanadium.

9. The composition of claim 1, wherein the drug is cisplatin, picoplatin, or a Pt(II) compound.

10. The composition of claim 1, wherein the composition has, as compared to the drug alone, at least one of:
(a) increased retention in blood circulation of the mammalian patient;
(b) reduced occurrence of accumulation in healthy tissues of organs of the mammalian patient; and
(c) increased occurrence of accumulation in tumors or malignant growths of the mammalian patient.

11. The composition of claim 1, wherein the composition comprises at least a first complex of a heparosan polymer with a first drug and a second complex of a heparosan polymer with a second drug.

12. A method of forming a composition, the method comprising the step of:
complexing a drug with a heparosan polymer to provide the composition of any of claim 1.

13. A method, comprising the step of:
administering a therapeutically effective amount of the composition of claim 1 to a mammalian patient so as to induce a therapeutic effect in the mammalian patient, wherein the drug is released from the heparosan polymer in the mammalian patient.

14. A method for increasing the half-life of a drug when administered to a mammalian patient, the method comprising the steps of:
complexing a drug with a heparosan polymer to provide the composition of claim 1; and
administering an effective amount of the composition to a mammalian patient so as to induce a therapeutic effect in the mammalian patient, wherein the heparosan polymer is biocompatible with the mammalian patient, and wherein the composition has, as compared to the drug alone, at least one of:
(a) increased retention in blood circulation of the mammalian patient;
(b) reduced occurrence of accumulation in organs of the mammalian patient; and
(c) increased occurrence of accumulation in tumors or malignant growths of the mammalian patient.
